(19) United States
(12) Patent Application Publication
(10) Pub. No.: US 2004/0180421 A1
(43) Pub. Date: Sep. 16, 2004

(54) Purification process

(76) Inventors: Jesus Zurdo, Cambridge (GB); Christopher Martin Dobson, Cambridge (GB)

Correspondence Address: Hamilton Brook, Smith & Reynolds, 530 Virginia Road, PO Box 9133, Concord, MA, 01742-9133, US

(21) Appl. No.: 10/482,138

(22) PCT Filed: Jun. 28, 2002
(86) PCT No.: PCT/GB02/03001

(30) Foreign Application Priority Data
Jun. 29, 2001 (GB) 0116022.5

(51) Int. Cl. C12P 21/06; C12N 9/64

(52) U.S. Cl. 435/226; 530/412; 435/68.1

(57) ABSTRACT

A method of purifying amyloid fibrils comprises treating a sample containing the fibris with a protease, detergent, chemical denatuarant or chaotrope. The amyloid fibrils may then be collected from the treated sample. The treatment is preferably carried out under conditions which allow digestion, degradation or denaturation of amorphous aggregates and soluble precursors in the sample but which do not digest, degrade or denature the amyloid fibrils. A homogonous preparation of amyloid fibrils is provided in which fibrils comprise at least 80% by weight of protein in the sample.

### FIELD OF THE INVENTION

The present invention relates to a method for the purification of fibrils and to preparations of amyloid fibrils essentially free of amorphous aggregates and soluble precursors.

### BACKGROUND OF THE INVENTION

Amyloid fibrils are highly organised proteinaceous aggregates associated with pathogenic disorders such as Alzheimer's disease or the transmissible spongiform encephalopatbies. Tan et al, Histopathology 1994 25, 403-414. They are also being explored as novel nanostructures with a wide variety of potential applications, Lashuel et al, Phil Trans R Soc Lond 2001, 256, 133-46.

Proteins susceptible to form amyloid fibrils do so from at least partially folded or destabilised states that lack the compact nature of the native protein. There is a constantly increasing number of proteins whose ability to form amyloid fibrils suggest amyloid formation is a common phenomenon and a generic property of polypeptide chains. One of such model proteins is the SH3 domain of the α-subunit of bovine phosphatidylinositol-3′-kinase (PI3-SH3), whose study has provided new insight in the processes driving amyloid fibril formation as well as details on the molecular packing of the protein chains within the amyloid fibrils. PI3-SH3 forms a partially folded state in solution at low pH, from which the protein slowly aggregates into fibrils with structural properties indistinguishable from those exhibited by amyloid fibrils related with human disease.

Despite the considerable advances in the structural characterisation of amyloid fibrils there are still a number of issues to be clarified. Among them is the detailed definition of the structural properties of the protein chains within the amyloid fibril. The main problem to do so is the intrinsic heterogeneity present in many amyloid fibril samples. Besides that, almost invariably both soluble precursors and non-fibrillar aggregates are present in all amyloid fibril samples formed in vitro. As a consequence, the structure adopted by the protein in the fibrils cannot be studied with a high certainty. The development of strategies to isolate amyloid fibrils from other species is, therefore, of valuable interest for the study of amyloid properties. Here we report a method to purify amyloid fibrils from other components. This approach allowed us to characterise the secondary structural elements featured by PI3-SH3 within the amyloid fibrils by FTIR methods avoiding contributions from soluble precursors and non-fibrillar aggregates.

### DESCRIPTION OF FIGURES

FIG. 1. Sensitivity of different PI3-SH3 aggregates to pepsin digestion monitored by FTIR. Samples containing mainly either amorphous aggregates (pH 1.5) or amyloid fibrils (pH 2.0) were incubated in the presence of pepsin at 37° C. in a ratio pepsin:PI3-SH3 1:200 in weight. Aliquots of both samples were taken at different times of incubation and FTIR spectra were recorded on them. A shift in the aggregation band towards higher wavenumbers together with a decrease of the component at 1684 cm⁻¹ (single arrow) and an increase between 1640 and 1660 cm⁻¹ (double arrow) are observed in the sample containing amyloid fibrils (pH 2.0).

FIG. 2. Characterisation of PI3-SH3 amyloid fibrils after purification. (A) FTIR spectra before (grey trace) and after (black trace) purification. The second derivative shows the main components present in each sample. (B) Curve fitting of amyloid fibrils before and after the purification procedure. Spectra were fitted to gaussian and lorentzian components. Components were found by second-derivative analysis. Despite the absence of a component at ˜1612 cm⁻¹ when analysing the digested sample by second derivative a better fit was achieved by artificially forcing such a component before the fitting routine. Curve fit shows a notorious decrease of components at ˜1684 and 1612 cm⁻¹ after pepsin digestion.

FIG. 3 shows the degree of protein solubilisation from HEWL amyloid fibrils at different denaturant concentrations.

### SUMMARY OF THE INVENTION

The present invention utilises differences in the stability of fibrils compared to other non-fibrillar contaminants in a sample, and in particular soluble precursor peptides and non-fibrillar aggregates in a sample.

The present invention provides a method of purifying a sample of amyloid fibrils comprising treating a sample containing fibrils with a protease, detergent, chemical denaturant or chaotrope and collecting fibrils from the treated sample.

In particular, the invention provides a method of purifying amyloid fibrils wherein the sample is treated with a protease, detergent, chemical denaturant or chaotrope under conditions which allow digestion or degradation of amorphous aggregates and soluble precursors present in the sample. In a preferred aspect of the present invention, the fibrils so produced are collected for example by centrifugation or filtration of the sample to isolate the amyloid fibril.

The invention also relates to a purified preparation of amyloid fibrils in which fibrils comprise at least 80%, preferably at least 85% or 90%, preferably at least 95% by weight of protein in the sample.

### DETAILED DESCRIPTION OF THE INVENTION

The present invention relates to a method of purifying amyloid fibrils. Samples of amyloid fibrils can be treated to reduce the heterogeneity present in the fibril sample. The present invention is particularly concerned with removing soluble precursors and non-fibrillar aggregates present in the fibril samples.

In accordance with the present invention, a method is provided to purify amyloid fibrils from a sample containing such fibrils. The present invention utilises differences in the stability of fibrils compared to other non-fibrillar components in the sample. In particular, the method is applicable to the removal of soluble precursor peptides and non-fibrillar aggregates in the sample. Preferably, a sample is treated with any agent to which the fibril is more stable than the corresponding soluble precursor or non-fibrillar aggregates. In particular, samples may be treated with agents which digest or degrade soluble precursors and amorphous aggregates or which denature such aggregates to facilitate isolation and purification of fibrils. Examples of agents which may be used in accordance with the present invention include proteases, detergents, chemical denaturants or chaotropes, organic solvents such as alcohols (TFE, BFIP, isopropanol), acetonitrile, chloroform, incubating the sample at high or low temperatures and the use of chemical agents such as cyanogen bromide. The conditions are selected such that they do not encourage formation of further fibrils to contaminate the initial fibril population. Suitable agents can be identified for each fibril sample, for example, by carrying out analysis on aliquots removed from the sample during the course of the incubation to assess whether there is a reduction in the presence of contaminants in the form of soluble precursors or non-fibrillar aggregates.

In preferred aspects, the method uses proteases, detergents, chemical denaturants or chaotropes to treat the sample. In a particularly preferred embodiment, proteases or other agents which degrade soluble peptides and non-fibrillar aggregates are provided, to degrade non-fibrillar peptides in the sample, while retaining the fibrils intact.

In accordance with the present invention, a method of purifying amyloid fibrils is provided which comprises treating a sample containing fibrils with a protease, detergent, chemical denaturant or chaotrope and collecting amyloid fibrils from the treated sample.

The fibril sample to be treated can be obtained by any suitable route. The fibrils may be fibrils formed from single or multiple peptide precursors, and so may comprise mixed fibrils. For example, the sample to be purified may be a sample isolated from a plaque formed in video. Fibril samples can be obtained from biopsies and tissue samples from patients and animals that present amyloid deposits containing polypeptides such as Aβ, IAPP, transthyretin, β2 microglobulin, apolipoprotein, AI, gelsolin, lysozyme, PrP or any other amyloidogenic protein. Alternatively, the sample to be treated could be one prepared by any suitable method in vitro, from protein samples subjected to conditions leading to fibril formation. For example fibril samples can be obtained from a polypeptide mentioned above, or a peptide or protein not related to disease, by incubating the polypeptide under at least partially denaturing conditions in the case of proteins and appropriate solution conditions in the case of small peptides through routine adjustment of parameters such as temperature, pH, ion strength, organic solvents, chemical denaturants and chelating agents.

The sample containing fibrils is treated with an agent in which the fibrils are more stable than peptide precursors, contaminents or amorphous aggregates in the sample. In one embodiment, the sample is treated with a protease. Any suitable protease may be used, such as, for example pepsin. Appropriate proteases can be selected, for example depending on the stability exhibited by the fibrils to purify under certain pH and temperature conditions. Pepsin has proteinase activity at low pH (optimal pH 1.8-2.5, 20-37° C.). Other proteases might be employed for similar purposes include: Trypsin (optimal pH 7.5-8.5, 20-37° C.), Pronase (optimal pH 6.0-8.0, 20-40° C.), Proteinase K (optimal pH 7.5-10.5, 20-37° C.), Papain (optimal pH 6.0-7.5, 20-37° C.), Elastase (optimal pH 7.5-8.5, 20-37° C.), Thrombin (optimal pH 7.5-9.0, 20-30° C.), Plasmin (optimal pH 8.0-9.5, 25° C.), etc.

Low specificity proteinases (Pepsine, Proteinase K, Papain, etc.) may preferably be used when the fibrillar material is highly resistant to digestion or when the fibrillar core is needed to be isolated for characterisation purposes, for example to identify the residues involved in the formation of the β-structure of the fibril and remove any other structural element from the fibrils, such as loops, helices, or disordered fragments. High specificity proteinases such as Plasmin, Factor-X Thrombin are preferably used when the fibrils are more sensitive to degradation or when the intact fibrillar material is needed, including loops and other structural elements or domains (functional or not) that do not participate in the β-structure core. Combinations of proteinases may also be used.

The sample may be contacted with the protease using any suitable method. Preferably, a sample is incubated with the suitable protease under conditions which allow digestion of amorphous aggregates and soluble precursors in the sample but which do not digest the amyloid fibrils. In, particular, the incubation conditions can be selected such that any protein contaminants in the sample are digested while not digesting the amyloid fibrils themselves. In preferred aspects of the invention, the sample is treated with the protease and subsequently incubated for sufficient time to essentially remove amorphous aggregates and soluble precursors or other contaminants from the sample.

The temperature of the incubation is selected based on the stability of the protease and amyloid fibrils under consideration. Suitable temperatures for treating the sample with protease include 20-60° C., preferably 30-40° C., such as 37° C. Protease may be added to the sample in any suitable quantity sufficient to cause digestion of the contaminants under the conditions of the treatment. For example, protease may be present in a ratio of about 1:1 or 1:10 to 1:10000 by weight of protease to protein in the sample, preferably about 1:20 to about 1:100. Selection of appropriate conditions is within the routine skill of those skilled in the art.

Suitably, the sample may be incubated for a period of time from 10 minutes, preferably 30 minutes up to 24 hours, 2 days, 3 days, depending on the quantity and stability of the contaminants to be removed.

Amyloid fibrils may also be purified by the use of detergents, chemical denaturants or chaotropic agents. Such agents may be used to denature peptides and to dissaggregate soluble and non-fibrillar peptides, to facillitate separation of fibrils from other peptide contaminants. Amyloid fibrils show in some cases a strong resistance to denaturation by anionic detergents such as SDS or LDS. Experiments performed on HEWL (hen egg white lysozyme) amyloid fibrils show that amyloid fibrils remain intact upon incubation in the presence of detergent concentrations up to about 1% w/v. Other non-ionic or cationic detergents may also be used with similar effects.

The presence of chemical denaturants or chaotropes such as urea or guanidine, in particular guanidine chloride or guanidinium thiocyanate affects to a different extent the monomeric protein, non-specific and amorphous aggregates and well-structured amyloid fibrils. Accordingly, chemical denaturants such as guanidine or urea may also be used. Guanidine may be used at a concentration of less than 4M, such as 1 to 4M. Suitable incubation conditions, such as temperature and length of incubation may be readily determined for each agent to be used. Suitable temperatures and incubation periods include those outlined above for proteases.

In one aspect of the invention, aliquots may be taken from the sample under treatment, fibrils separated from such aliquots and suitable analysis such as FTIR (fourier transform infrared spectroscopy) analysis, SDS-PAGE analysis or electron microscopy or electrospray mass spectrometry to assess the levels of amorphous aggregates or other contaminants present in the sample. Such an analysis may be used to assess the preferred treatment conditions to obtain purified fibrils.

Prior to separation, for example by centrifugation or filtration, or further analysis, the protease in the sample may be inactivated, for example by addition of appropriate protease inhibitors. For example pepsin or other proteases maybe inactivated by washing in ammonium carbonate or Tris pH 8.0. Other protease inhibitors include PMSF (for example 1 mM), benzamidine (for example 1 mM), leupeptin (for example 10 μg ml⁻¹), pepstatin (for example 10 μg ml⁻¹), aprotinin (for example 10 μg ml⁻¹), antipain (for example 10 μg ml⁻¹) EDTA (for example 10 μg ml⁻¹), etc.

Following treatment with protease, detergent or other agent amyloid fibrils can be separated from the sample by any suitable method. Typical isolation procedures include filtration, for example through a pore-controlled membrane and centrifugation (sedimentation). In some cases however, functional groups in the surface of the fibrils may be used for that purpose (e.g. sulphydril groups, ligands—DNA oligos-, etc.) by binding the fibrils to active surfaces (e.g. gold surfaces in the case of sulphydril groups, etc.) and washing. Chemical cross-linkers could also be used, for example to react preferentially with soluble or non-fibrillar aggregates and facilitate separation. Affinity of fibrils versus soluble molecules or small aggregates should be greatly stabilised due to the large amount of active groups present in the fibril surface. Preferably, centrifugation or filtration is used with subsequent collection of the fibril components. For example, the sample may be centrifuged between 100,000-500,000 g preferably 300,000 g for a period from 15 mins to 4 hours, such as 1 hour. After discarding the supernatant, the pellet may be collected, resuspended in suitable medium, and the centrifugation procedure repeated. Suitable washes may also be carried out.

The sample of purified fibrils may be collected for example by collecting the pellet obtained by centrifugation. Such fibrils may be resuspended or manipulated for further investigation or use, for example for carrying our further studies such as FTIR, electron microscopy, electrospray mass spectrometry and SDS-PAGE, or other suitable methods to investigate fibril structure such as hydrogen exchange monitored by mass spectrometry and NMR. Alternatively fibrils may be manipulated for use in nanostructures.

Purified fibrils may be used for identification of key residues involved in aggregation. Analysis of key residues that participate in aggregation in pathological proteins can be performed only if highly pure fibrils are obtained (Hoshino et al. Nature Struct. Biology. 2002, 9: 332-336). Identification of these residues involved in aggregation can be essential in the design of drugs to prevent aggregation or in the engineering or new variants of proteins resistant to aggregation in both disease-related proteins and bio-active peptides and proteins used as drugs or in the industry.

Amyloid fibrils may also be purified for their use as nanostructures and nanomaterials. Purified fibrils can be then spun into silk-like fibres, used as nanowires (taking advantage of their conductive properties), used as matrix for cell growth, use as plastic bio-compatible and bio-degradable material in implants, used as encapsulating material for the delivery of drugs, used as a coating agent (paint) to confer specific properties to surfaces (water-repellent, antifouling surfaces), modify properties of surfaces (confer optical properties, such as fluorescent emission, etc) to be used as sensors, etc.

### EXAMPLE

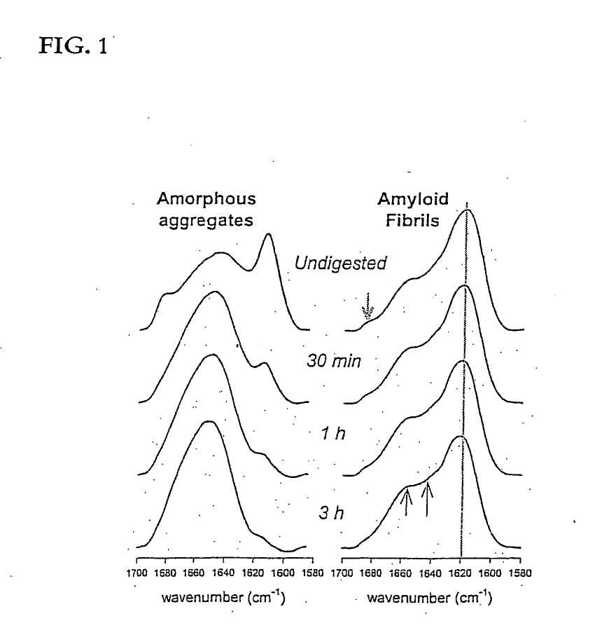

Samples of amyloid fibrils prepared from PI3-SH3 are observed to contain at least some quantities of both non-aggregated and non-fibrillar material. Separation of fibrils from other species, especially big aggregates, is difficult to achieve by common biochemical approaches due to the similarities in their properties and large size. To overcome this problem, a new approach based on proteinase digestion was attempted; since both kinds of aggregates seem to show different structural characteristics they would likely exhibit different sensitivity to proteinase digestion. For this purpose pepsin was chosen due to its non-specific proteinase activity at low-pH. Samples of two different kinds of PI3-SH3 aggregates were prepared as described elsewhere. One type of samples incubated at pH 1.5 contained amorphous aggregates, whereas the other, incubated at pH 2.0, contained well-defined amyloid fibrils. Aggregate digestion was monitored by FTIR at different times of incubation (FIG. 1). Spectral features associated to amorphous aggregates formed at pH 1.5 show a quick disappearance of their β-sheet-related components (1612 and 1684 cm⁻¹ respectively) to render a main peak at ca. 1649 cm⁻¹ related to non-structured conformations. By contrast the spectrum corresponding to the sample containing amyloid fibrils (incubated at pH 2.0) show very little evolution with time as the main β-sheet spectral features associated with aggregation suffer only small changes even after 3 hours of incubation. Interestingly a decrease in the intensity of the band ca. 1684 cm⁻¹ can be observed together with a small shift to higher wavenumbers by the main peak centered at ca 1618 cm⁻¹. This could be indicative of digestion of some of the aggregated species present in the sample, which contribute at 1684 and 1612 cm⁻¹ respectively. These changes suggest that the amorphous species present in the sample are digested to leave just the protease resistant fibrils. There is also an increase in intensity between 1640 and 1660 cm⁻¹, due to a higher population of disordered species (digested peptides), i.e. peptide fragments.

The integrity of the amyloid fibrils after pepsin digestion was analysed by different biophysical techniques. Micrographs obtained by electron microscopy (EM) analysis of the samples showed abundant intact amyloid fibrils after pepsin digestion which retain the overall morphology evidenced in the original preparations, whereas other types of aggregates were undetectable. The integrity of the protein molecule within the amyloid fibrils was assessed by recording 1D-NiMR spectra of PI3-SH3 after amyloid fibril disruption using 6M Gnd HCl and refolding procedures. The spectra were indistinguishable from that of the native protein prior to aggregation. SDS-PAGE of PI3-SH3 from fibrils before and after treatment with pepsin, lanes were loaded as follows. Lane 1: native protein; lane 2: fibrils before addition of pepsin; lane 3: pellet obtained after digestion of sample in lane 2 by incubation with pepsin; lane 4: supernatant obtained after digesting sample in lane 2 with pepsin. A minor band appears below the monomer band in lane 2, probably due to a small amount of protein degradation after the long times of incubation at low pH required for amyloid fibril formation. This band is completely absent in lane 3, and therefore in the amyloid fibrils. Interestingly SDS-PAGE of samples containing PI3-SH3 amyloid fibrils show a band migrating at the same position that the PI3-SH3 monomer, indicating that the fibrils are formed by the intact full-length protein. Samples containing fibrils that underwent pepsin digestion and ultracentrifugation to eliminate any soluble component show again that only the full-length polypeptide is present and that no other protein fragment can be detected. By contrast, the supernatant obtained after such procedure shows no observable band, indicating that any soluble monomer present in the original sample was completely degraded.

Electrospray Mass Spectrometry (EMS) analysis of amyloid fibril samples after pepsin digestion and subsequent ultra-centrifugations yielded masses correspondent to the full length PI3-SH3 domain, whereas no other smaller fragment was detected. Therefore, SDS-PAGE and EMS analysis show that the morphological and spectroscopic characteristics exhibited by the fibrils after the cleaning procedure correspond exclusively to full length PI3-SH3 incorporated into the well-ordered amyloid fibrils, most likely because PI3-SH3 adopts a very compact conformation in the fibrils that makes it inaccessible to the proteinase.

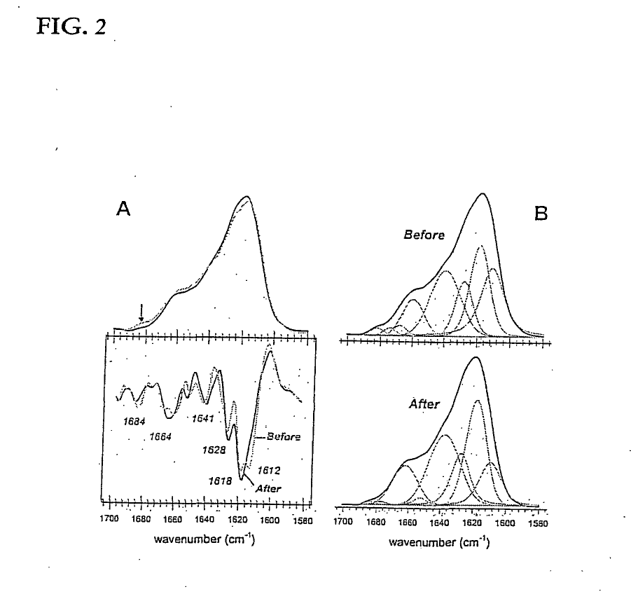

Once we confirmed that amyloid fibrils are proteinase resistant and that, by contrast, amorphous aggregates and soluble protein still present in the original sample are not, we proceeded to further characterise the structural features exhibited by amyloid fibrils using FTIR. FTIR spectra of the original and purified samples show an overall similarity (FIG. 2), indicating that most of the contributions to the amide I band of the original sample come from the fibrils present in the solution. However, a detailed analysis of the spectra reveals some differences, evident particularly when the second derivatives of both spectra are compared. The component at 1684 cm⁻¹ is virtually missing after digestion, and one of the three components of the main aggregation band, that at 1612 cm⁻¹, is absent by second derivative analysis (FIG. 2). This suggests that the bands at 1684 and 1612 cm⁻¹, the main components in samples containing amorphous aggregates (see FIG. 1), are characteristic of non-fibrillar material present in the sample before the purification procedure.

We can then assume that the hydrogen bonding components found in the amyloid fibrils are principally 1618 and 1628 cm⁻¹ corresponding to characteristic β structure. Other components at 1641 and 1664 cm⁻¹ are still present in the fibrils after digestion indicating some contribution from disordered conformations and β-turns respectively which indicates that apart from the main β-sheet features some regions of the protein could display flexible loops and turns within the amyloid fibrils. This latter conclusion is consistent with the model of the SH3 fibrils based on cryo-EM analysis but is remarkable given the lack of susceptibility of the protein in the fibrils to proteolysis. It must reflect the fact that even those regions of the polypeptide chains not in well-defined β-sheet structures are tightly held within the fibrillar assemblies.

As well as providing evidence for different types of secondary structure, FTIR analysis is reported to distinguish between parallel and anti-parallel configurations in β-sheets. It is possible, however, that the specific geometry of the β-strands in the fibrils could perturb the IR frequencies from those classical of β-sheet structures.

FTIR analysis of the purified amyloid fibrils shows the notoriously decreased contribution at 1611-1612 cm⁻¹ and virtually no contribution at 1684 cm⁻¹ (FIG. 2). This observation strongly suggests that these decreased components are associated to non-fibrillar aggregates, in agreement with the spectral features observed for samples incubated at much lower pH values containing amorphous aggregates (FIG. 1). The almost complete absence in the purified fibrils of components at ca. 1684 cm⁻¹ suggests an almost inexistent contribution of antiparallel relative to parallel interactions. These results indicate that extreme care must be taken in the analysis of amyloid fibrils by techniques such as FTIR to ensure that signals from disordered and soluble material do not contribute to the spectra. Further FTIR studies of purified amyloid fibrils are required to confirm the parallel and/or antiparallel nature of the β-sheets and their connection with defined aggregate/fibril structures and morphologies. Interestingly, studies of highly fibrillar samples of insulin have also suggested a parallel β-sheet structure, as has solid-state NMR and FITR in some samples of amyloid fibrils. However, there is still no certainty about the required conformation of the β-structure within amyloid or its relation to precise fibril morphologies, since also antiparallel structure has been reported for some other amyloids, whereas other authors yet postulate that a mixture parallel-antiparallel could coexist. Further studies of highly purified materials are therefore needed to establish conclusively the orientation of the β-strands in amyloid fibrils. The ability to study fibrillar material, generated by approaches such as that we report here, should be of particular importance in these studies.

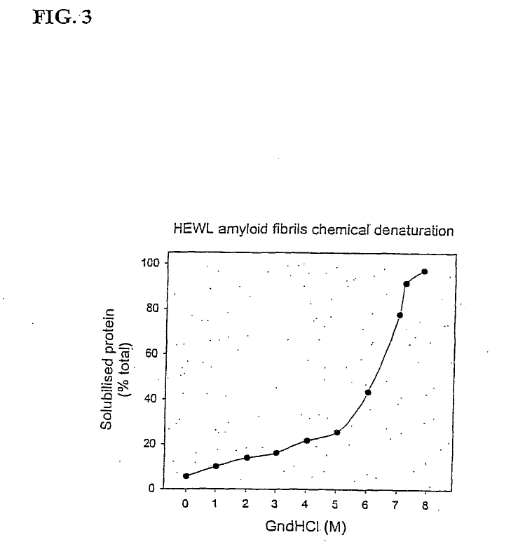

To evaluate the resistance of amyloid fibrils to chemical denaturation, aliquots of fibrillated sample of HEWL were incubated in the presence of different concentrations of guanidine. The amount of protein solubilised by the treatment was evaluated after sedimentation of these samples using a centrifugal force of 300,000 g for 1 hour. The supernatant obtained was measured at 280 nm. At the same time the pellet obtained was analysed by electron microscopy. The results are shown in FIG. 3.

Analysis of the pellets obtained after incubation with guanidine at concentrations up to 4M show large amounts of amyloid fibrils with similar morphologies to those exhibited by untreated samples. However some protein appears to be solubilised at intermediate concentrations of guanidine (1-4M), which suggests that some non-specific aggregates of loosely bound protein molecules are being removed by the treatment. This suggests that the resulting fibrils after treatment with such intermediate concentrations of denaturant are ‘cleaner’ than the starting samples.

In conclusion, amyloid fibrils free of amorphous aggregates and soluble precursors can be prepared by using proteolytic digestion, ultra-centrifugation detergents and chemical denaturants. This approach has enabled us to demonstrate that the full-length protein is preserved within the SH3 fibrils studies here. FTIR analysis shows that the protein within the fibrils has FTIR bands typical of a β-sheet structure that is mainly parallel in character. Other regions of the polypeptide chain appear to form turns and disordered structures that are likely to link the β-strands, although these seem to be tightly held within the fibril structure. The approach described here should be applicable to a wide range of other amyloid systems, and to be important not only to investigate disease-related aggregates but also as a means of preparing and characterising novel materials assembled from protein fibrils.

Methods

Amorphous aggregates were prepared by incubating a 0.5 mM solution of PI3-SH3 in ²H₂O at pH 1.5 for 5 days at 35° C. Amyloid fibrils were prepared by incubating a 0.5 mM solution of PI3-SH3 in ²H₂O at pH 2.0 for 30 days at 35° C.

Samples were characterised by EM before proteinase digestion to confirm their morphology. Sample incubated at pH 1.5 for 6 days at 35° C. showed non-fibrillar aggregates, whereas the sample incubated at pH 2.0 for 30 days at 35° C. showed well-defined fibrils. Both samples were prepared in ²H₂O, and readings were corrected for isotope effect.

Protein digestion was carried out by incubating both samples with pepsin in a ratio pepsin:PI3-SH3 1:200 in weight at 37° C. Aliquots were taken at different times and analysed by FTIR without further processing.

Samples of amyloid fibrils were incubated in 6 M GndHCl which immediately solubilised the fibrils. Dialysis in 20 mM sodium phosphate buffer pH 7.2 was carried out to remove the GndHCl and refold the protein to its native conformation. 1D NMR proton spectra of the recovered protein from fibrils and freshly prepared protein in the same buffer were recorded.

Incubation of fibrils in the presence of 1% SDS or LDS used as loading buffers in the electrophoresis completely solubilised the amyloid fibrils. Fibrils were dissociated by incubating samples in 10% ammonium hydroxide before MS analysis.

Samples containing amyloid fibrils prepared by incubating a 1 mM PI3-SH3 solution in ²H₂O at pH 2.0 and incubating it for 30 days at 35° C. were digested with pepsin in a ration pepsin:PI3-SH3 (1:100) in weight for 3 hours at 37° C. and then subjected to ultra-centrifugation for 1 h at 300,000 g. The pellet containing the undigested fibrils was then resuspended in ²H₂O pH 2.0 and centrifuged again in identical conditions. The pellet was again resuspended in ²H₂O pH 2.0 and then analysed by FTIR together with an aliquot of the original sample before the purification procedure.

Jiménez, J. L.; Guijarro, J. L.; Orlova, E.; Zurdo, J.; Dobson, C. M.; Sunde, M.; Saibil, H. R. EMBO J 1999, 18, 815-821.

Zurdo, J.; Guijarro, J. I.; Jiménez, J. L.; Saibil, H. R.; Dobson, C. M. J. Mol Biol (submitted) 2001.

Krimm, S.; Bandekar, J. Adv Protein Chem 1986, 38, 181-364. Fabian, H.; Choo, L. P.; Szendrei, G. I.; Jackson, M.; Halliday, W. C.; Otvos, L.; Mantsch, H. H. Appl Spectrosc 1993, 47,1513-1518.

Bouchard, M.; Zurdo, J.; Nettleton, E. J.; Dobson, C. M.; Robinson, C. V. Protein Sci 2000, 9, 1960-1967.

Benzinger, T. L. S.; Gregory, D. M.; Burkoth, T. S.; Miller-Auer, H.; Lynn, D. G.; Bofto, R. E.; Meredith, S. C. Proc Natl Acad Sci USA 1998, 95, 13407-13412.

Antzutkin, O. N.; Balbach, J. J.; Leapman, R D.; Rizzo, N. W.; Reed, J.; Tycko, R. Proc Natl Acad Sci USA 2000, 97, 13045-13050.

Lansbury, P. T., Jr.; Costa, P. R.; Griffiths, J. M.; Simon, E. J.; Auger, M.; Halverson, K. J.; Kocisko, D. A.; Hendsch, Z. S.; Ashbum, T. T.; Spencer, R. G.; et al. Nat Struct Biol 1995, 2, 990-8. Villegas, V.; Zurdo, J.; Filimonov, V. V.; Aviles, F. X.; Dobson, C. M.; Serrano, L. Protein Sci 2000, 9, 1700-1708.

Sunde, M.; Blake, C. Adv Protein Chem 1997, 50, 123-59.

Stone M. J. Blood 1990 75, 531-545, Nordstedt et al, Amyloid 2000, 7, 212-7.

Balbirnic et al Proc Natl. Acad. Sci 2001, 98, 2375-80.

1. A method of purifying amyloid fibrils comprising (a) treating a sample containing fibrils with a protease, detergent, chemical denaturant or chaotrope and (b) collecting amyloid fibrils from the treated sample:

2. A method according to claim 1 wherein step (a) comprises treating said sample with a protease, detergent, chemical denaturant or chaotrope under conditions to allow digestion, degradation or denaturation of amorphous aggregates and soluble precursors in the sample, but which do not digest, degrade or denature amyloid fibrils.

3. A method according to claim 1 wherein step (a) comprises treating the sample with a protease and incubating the sample from 30 minutes to 24 hours.

4. A method according to claim 1 wherein step (a) comprises treating the sample in the presence of a protease in a temperature range of 20-60 ° C.

5. A method according to claim 1 wherein step (a) comprises treating the sample with a protease, and the protease is present in a ratio of 1:10 to 1:10000 protease to protein by weight.

6. A method according to claim 1 wherein step (a) comprises treating the sample with a protease, selected from pepsin, trypsin, pronase, proteinase K, papin, elastase, thrombin or plasmin.

7. A method according to claim 6 wherein the protease is pepsin.

8. A method according to claim 1 wherein step (a) comprises treating the sample with a detergent selected from an anionic, non-ionic or cationic detergent.

9. A method according to claim 1 wherein step (a) comprises treating the sample with a chemical denaturant or chaotrope selected from urea or guanidine.

10. A method according to claim 1 wherein step (b) comprises subjecting the treated sample to centrifugation or filtration and thereby collecting the amyloid fibrils.

11. A homogenous preparation of amyloid fibrils, said fibrils comprising at least 80% by weight of protein in the sample.

12. A homogenous preparation of fibrils according to claim 11 wherein said fibrils comprise at least 95% by weight of protein in the sample.

13. A purified preparation of amyloid fibrils obtainable by the method of claim 1.
